(19) United States
(12) Patent Application Publication
(10) Pub. No.: US 2022/0081477 A1
(43) Pub. Date: Mar. 17, 2022

(54) CONTROLLED FUCOSYLATION OF ANTIBODIES

(71) Applicant: Seagen Inc., Bothell, WA (US)

(72) Inventors: Shanta BODDAPATI, Bothell, WA (US); Aaron CHEN, Bothell, WA (US); Swapnil BHARGAVA, Bothell, WA (US)

(21) Appl. No.: 17/309,778

(22) PCT Filed: Dec. 18, 2019
(86) PCT No.: PCT/US2019/067222

§ 371 Date: Jun. 17, 2021

Related U.S. Application Data
(60) Provisional application No. 62/781,691, filed on Dec. 19, 2018.

(51) Int. Cl. C07K 16/00

(52) U.S. Cl. CPC C07K 16/00; C07K 2317/14; C07K 2317/41

(57) ABSTRACT

The invention provides methods for preparing antibodies and antibody derivatives with controlled levels of core fucosylation. In one aspect, provided herein is a method of controlling the level of afucosylation of an antibody or antibody derivative. In some embodiments, the invention provides a composition of antibodies or antibody derivatives produced by the instant methods. The antibodies and derivatives can be formulated as pharmaceutical compositions comprising a therapeutically or prophylactically effective amount of the antibody or derivative and one or more pharmaceutically acceptable ingredients.

### CROSS REFERENCE TO RELATED APPLICATIONS

This application claims the benefit of priority of U.S. Provisional Application No. 62/781,691, filed Dec. 19, 2018, which is incorporated by reference in its entirety for all purposes.

### BACKGROUND OF THE INVENTION

Recombinant therapeutic proteins are produced by many different methods. One preferred method is production of recombinant proteins from mammalian host cell lines. Cell lines, such as Chinese Hamster Ovary (CHO) cells, are engineered to express the therapeutic protein of interest. Different cell lines have advantages and disadvantages for recombinant protein production, including protein characteristics and productivity. Selection of a cell line for commercial production often balances the need for high productivity with the ability to deliver consistent product quality with the attributes required of a given product. One important class of therapeutic recombinant proteins that require consistent, high quality characteristics and high titer processes are monoclonal antibodies.

Monoclonal antibodies produced in mammalian host cells can have a variety of post-translational modifications, including glycosylation. Monoclonal antibodies, such as IgG1s, have an N-linked glycosylation site at asparagine 297 (Asn297) of each heavy chain (two per intact antibody). The glycans attached to Asn297 on antibodies are typically complex biantennary structures with very low or no bisecting N-acetylglucosamine (bisecting GIcNAc) with low amounts of terminal sialic acid and variable amounts of galactose. The glycans also usually have high levels of core fucosylation. Reduction of core fucosylation in antibodies has been shown to alter Fc effector functions, in particular Fcgamma receptor binding and ADCC activity. This observation has lead to interest in the engineering cell lines so they produce antibodies with reduced core fucosylation.

Methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knock out gene expression entirely. Alternatives to engineering cell lines include the use of small molecule inhibitors that act on enzymes in the glycosylation pathway.

In some applications, it may be desirable to use a mixed population of core fucosylated and core afucosylated antibodies or antibody derivatives. As such, improved methods for predicting culture parameters that will result in a desired level of core afucosylation are needed.

### BRIEF SUMMARY OF THE INVENTION

The invention provides methods for preparing antibodies and antibody derivatives with controlled levels of core afucosylation. The methods are premised in part on the unexpected results presented in the Examples showing that accurate predictive models of antibody or antibody derivative afucosylation levels with a given fucosylation inhibitor (e.g., 2-fluorofucose) can be generated using parameters related to the fucosylation inhibitor (e.g., amount of fucosylation inhibitor or time of fucosylation inhibitor addition) paired with a corresponding culture parameter (e.g., integral of cell area or antibody titer) as inputs to the predictive model.

In one aspect, provided herein is a method of controlling the level of afucosylation of an antibody or antibody derivative, comprising: (a) culturing a host cell in a culture medium in the presence of a pre-determined amount of an inhibitor of fucosylation (Aₚ), wherein the host cell expresses an antibody or antibody derivative having an Fc domain having at least one complex N-glycoside-linked sugar chain bound to the Fc domain through an N-acetylglucosamine of the reducing terminal of the sugar chain; and (b) isolating the antibody or antibody derivative, wherein Aₚ is pre-determined such that the level of afucosylation of the isolated antibody or antibody derivative of (b) has a level of afucosylation that does not exceed a maximum deviation from a target level of afucosylation. In some embodiments, the antibody or antibody derivative is isolated upon completion of culturing. In some embodiments, the method further comprises determining Ap. In some embodiments,

In some embodiments, according to any of the methods of controlling the level of afucosylation of an antibody or antibody derivative employing Ap described above, Ap is determined based on a predictive model generated using a plurality of different fucosylation inhibitor amounts and a cell growth parameter of the host cell in the culture as inputs and the level of afucosylation of the isolated antibody or antibody derivative as the output. In some embodiments, the predictive model is generated using fucosylation inhibitor amounts normalized to the cell growth parameter as inputs. In some embodiments, the cell growth parameter is integral cell area (ICA). In some embodiments, the method further comprises comprising generating the predictive model.

In another aspect, provided herein is a method of controlling the level of afucosylation of an antibody or antibody derivative, comprising: (a) culturing a host cell in a culture medium, wherein the host cell expresses an antibody or antibody derivative having an Fc domain having at least one complex N-glycoside-linked sugar chain bound to the Fc domain through an N-acetylglucosamine of the reducing terminal of the sugar chain; (b) adding a saturating amount of an inhibitor of fucosylation to the culture medium at a pre-determined time (Tp) during the culturing, wherein the saturating amount of the fucosylation inhibitor results in at least about 95% afucosylation when added at d0 of the culturing; and (c) isolating the antibody or antibody derivative, wherein Tp is pre-determined such that the level of afucosylation of the isolated antibody or antibody derivative of (c) has a level of afucosylation that does not exceed a maximum deviation from a target level of afucosylation. In some embodiments, the antibody or antibody derivative is isolated upon completion of culturing. In some embodiments, the method further comprises determining Tp.

In some embodiments, according to any of the methods of controlling the level of afucosylation of an antibody or antibody derivative employing Tp described above, Tp is determined based on a predictive model generated using titer of the antibody or antibody derivative in the culture at a plurality of different saturating fucosylation inhibitor addition times in the culturing as inputs and the level of afucosylation of the isolated antibody or antibody derivative as the output. In some embodiments, the method further comprises generating the predictive model.

In some embodiments, according to any of the methods of controlling the level of afucosylation of an antibody or antibody derivative described above, the fucosylation inhibitor is a fucose analog. In some embodiments, the fucose analog is 2-fluorofucose (2FF), the compound of formula I, or the compound of formula II. In some embodiments, the fucose analog is 2FF.

In some embodiments, according to any of the methods of controlling the level of afucosylation of an antibody or antibody derivative described above, the target level of afucosylation is: (a) about 100% to about 90%; (b) about 90% to about 80%; (c) about 80% to about 70%; (d) about 70% to about 60%; (e) about 60% to about 50%; (f) about 50% to about 40%; (g) about 40% to about 30%; (h) about 30% to about 20%; (i) about 20% to about 10%; or (j) about 10% to about 0%.

In some embodiments, according to any of the methods of controlling the level of afucosylation of an antibody or antibody derivative described above, the target level of afucosylation is: (a) greater than about 80%; (b) greater than about 60%; (c) greater than about 40%; (d) greater than about 20%; (e) greater than about 10%; or (f) greater than about 5%.

In some embodiments, according to any of the methods of controlling the level of afucosylation of an antibody or antibody derivative described above, the maximum deviation from the target level of afucosylation is no more than 10%. In some embodiments, the maximum deviation from the target level of afucosylation is no more than 5%.

In some embodiments, according to any of the methods of controlling the level of afucosylation of an antibody or antibody derivative described above, the host cell is a recombinant host cell. In some embodiments, the host cell is a Chinese hamster ovary (CHO) cell.

In some embodiments, according to any of the methods of controlling the level of afucosylation of an antibody or antibody derivative described above, the host cell is a hybridoma.

In some embodiments, according to any of the methods of controlling the level of afucosylation of an antibody or antibody derivative described above, the host cell is grown in fed batch culture.

In some embodiments, according to any of the methods of controlling the level of afucosylation of an antibody or antibody derivative described above, the host cell is grown in continuous feed culture.

In some embodiments, according to any of the methods of controlling the level of afucosylation of an antibody or antibody derivative described above, the culture medium has a volume of at least 100 liters. In some embodiments, the culture medium has a volume of at least 500 liters.

In some embodiments, according to any of the methods of controlling the level of afucosylation of an antibody or antibody derivative described above, the culture media is an animal protein free media.

In some embodiments, according to any of the methods of controlling the level of afucosylation of an antibody or antibody derivative described above, isolating the antibody or antibody derivative comprises isolating the antibody or antibody derivative from the cell and/or the culture medium. In some embodiments, isolating the antibody or antibody derivative comprises using a protein A column. In some embodiments, isolating the antibody or antibody derivative comprises using a cation or anion exchange column or a hydrophobic interaction column.

In some embodiments, according to any of the methods of controlling the level of afucosylation of an antibody or antibody derivative described above, the antibody or antibody derivative is an intact antibody. In some embodiments, the intact antibody is an IgG1 antibody.

In some embodiments, according to any of the methods of controlling the level of afucosylation of an antibody or antibody derivative described above, the antibody or antibody derivative is a single chain antibody.

In some embodiments, according to any of the methods of controlling the level of afucosylation of an antibody or antibody derivative described above, the antibody or antibody derivative comprises a heavy chain variable region, a light chain variable region, and an Fc region.

In some embodiments, according to any of the methods of controlling the level of afucosylation of an antibody or antibody derivative described above, the antibody or antibody derivative is an antibody derivative comprising an antibody Fc region and a ligand binding domain of a non-immunoglobulin protein.

These and other aspects of the present invention may be more fully understood by reference to the following detailed description, non-limiting examples of specific embodiments, and the appended figures.

### BRIEF DESCRIPTION OF THE DRAWINGS

FIG. 1A shows a viable cell density (VCD) curve showing growth of a cell line treated with various concentrations of 2-fluorofucose (2FF).

FIG. 1B shows a saturation model for % afucosylation as a function of [2FF concentration]/ICA combining multiple CHO cell lines with different growth characteristics.

FIG. 2A shows the average titer for two different cell lines treated with 2FF on a delayed schedule.

FIG. 2B shows a comparison of afucosylation for control, day 0 (d0) addition, and day 3 (d3) addition of 2FF (all days counted from start of production) for cell line A.

FIG. 2C shows a comparison of afucosylation for control, day 0 (d0) addition, and day 3 (d3) addition of 2FF (all days counted from start of production) for cell line B.

### DETAILED DESCRIPTION OF THE INVENTION

### Definitions

The term “antibody” refers to (a) immunoglobulin polypeptides and immunologically active portions of immunoglobulin polypeptides, i.e., polypeptides of the immunoglobulin family, or fragments thereof, that contain an antigen binding site that immunospecifically binds to a specific antigen (e.g., CD70) and an Fc domain comprising a complex N-glycoside-linked sugar chain(s), or (b) conservatively substituted derivatives of such immunoglobulin polypeptides or fragments that immunospecifically bind to the antigen (e.g., CD70). Antibodies are generally described in, for example, Harlow & Lane, Antibodies: A Laboratory Manual (Cold Spring Harbor Laboratory Press, 1988). Unless otherwise apparent from the context, reference to an antibody also includes antibody derivatives as described in more detail below.

An “antibody derivative” means an antibody, as defined above (including an antibody fragment), or Fc domain or region of an antibody comprising a complex N-glycoside linked sugar chain, that is modified by covalent attachment of a heterologous molecule such as, e.g., by attachment of a heterologous polypeptide (e.g., a ligand binding domain of heterologous protein), or by glycosylation (other than core fucosylation), deglycosylation (other than non-core fucosylation), acetylation, phosphorylation or other modification not normally associated with the antibody or Fc domain or region.

The term “monoclonal antibody” refers to an antibody that is derived from a single cell clone, including any eukaryotic or prokaryotic cell clone, or a phage clone, and not the method by which it is produced. Thus, the term “monoclonal antibody” is not limited to antibodies produced through hybridoma technology.

The term “Fc region” refers to the constant region of an antibody, e.g., a C_{H}1-hinge-C_{H}2-C_{H}3 domain, optionally having a C_{H}4 domain, or a conservatively substituted derivative of such an Fc region.

The term “Fc domain” refers to the constant region domain of an antibody, e.g., a C_{H}1, hinge, C_{H}2, C_{H}3 or C_{H}4 domain, or a conservatively substituted derivative of such an Fc domain.

An “antigen” is a molecule to which an antibody specifically binds.

The terms “specific binding” and “specifically binds” mean that the antibody or antibody derivative will bind, in a highly selective manner, with its corresponding target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The terms “inhibit” or “inhibition of” means to reduce by a measurable amount, or to prevent entirely.

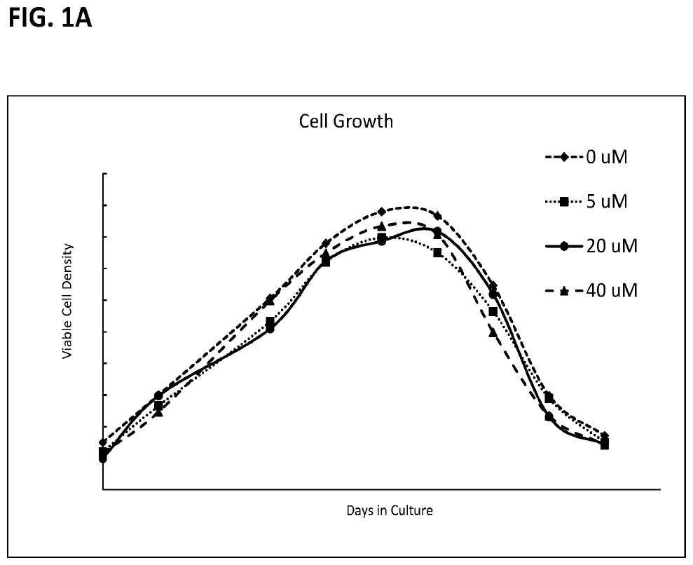

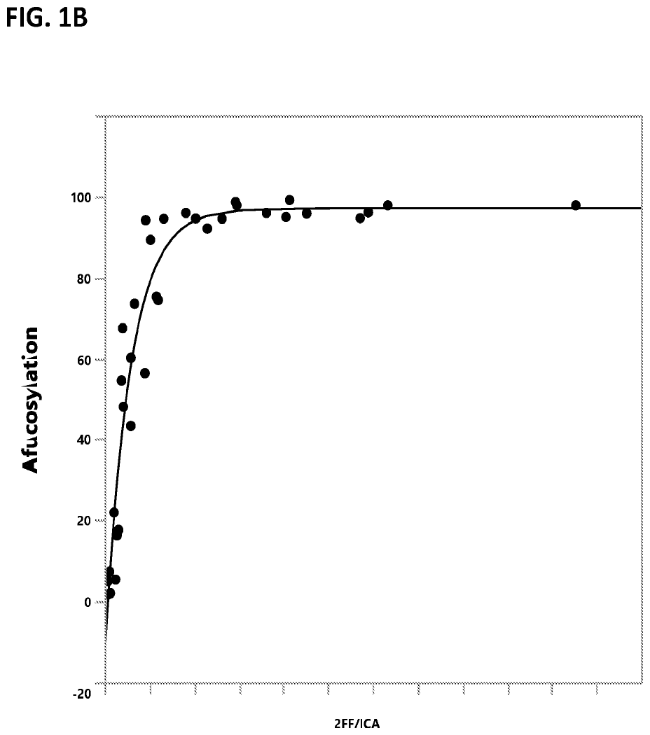

The term “integral cell area” or “ICA” represents the area under the viable cell density (VCD) curve for a given cell line as shown in FIG. 1A and can be calculated, for example, by integrating the VCD curve.

The term “pharmaceutically acceptable” means approved by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which the antibody or antibody derivative is administered.

The term “biologically acceptable” means suitable for use in the culture of cell lines for the manufacture of antibodies. Exemplary biologically acceptable salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene bis-(2 hydroxy 3-naphthoate)) salts. A biologically acceptable salt may involve the inclusion of another molecule such as an acetate ion, a succinate ion or other counterion. The counterion may be any organic or inorganic moiety that stabilizes the charge on the parent compound. Furthermore, a biologically acceptable salt may have more than one charged atom in its structure. Instances where multiple charged atoms are part of the biologically acceptable salt can have multiple counter ions. Hence, a biologically salt can have one or more charged atoms and/or one or more counterion.

Therapeutic agents of the invention are typically substantially pure from undesired contaminant. This means that an agent is typically at least about 50% w/w (weight/weight) purity, as well as being substantially free from interfering proteins and contaminants. Sometimes the agents are at least about 80% w/w and, more preferably at least 90% or about 95% w/w purity. Using conventional protein purification techniques, homogeneous peptides of at least 99% w/w can be obtained.

As used herein, “alkynyl fucose peracetate” refers to any or all forms of alkynyl fucose (5-ethynylarabinose) with acetate groups on positions R¹⁻⁴ (see formula I and II, infra), including 6-ethynyl-tetrahydro-2H-pyran-2,3,4,5-tetrayl tetraacetate, including the (2S,3S,4R,5R,6S) and (2R,3S,4R,5R,6S) isomers, and 5-((S)-1-hydroxyprop-2-ynyl)-tetrahydrofuran-2,3,4-triyl tetraacetate, including the (2S,3S,4R,5R) and (2R,3S,4R,5R) isomers, and the aldose form, unless otherwise indicated by context. The terms “alkynyl fucose triacetate”, “alkynyl fucose diacetate” and “alkynyl fucose monoacetate” refer to the indicated tri-, di- and mono-acetate forms of alkynyl fucose, respectively.

Unless otherwise indicated by context, the term “alkyl” refers to a substituted or unsubstituted saturated straight or branched hydrocarbon having from 1 to 20 carbon atoms (and all combinations and subcombinations of ranges and specific numbers of carbon atoms therein), with from 1 to 3, 1 to 8 or 1 to 10 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl.

Alkyl groups, whether alone or as part of another group, can be optionally substituted with one or more groups, preferably 1 to 3 groups (and any additional substituents selected from halogen), including, but not limited to: halogen, —θ(C₁-C₈ alkyl), —O—(C₂-C₈ alkenyl), —O—(C₂-C₈ alkynyl), aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —NH₂, —NH(R′), —N(R′)₂ and —CN; where each R′ is independently selected from —H, —C₁-C₈ alkyl, —C₂-C₈ alkenyl, —C₂-C₈ alkynyl, or aryl. In some embodiments, the —O—(C₁-C₈ alkyl), —O—(C₂-C₈ alkenyl), —O—(C₂-C₈ alkynyl), aryl, and R′ groups can be further substituted. Such further substituents include, for example, —C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —NH₂, —NH(R″), —N(R″)₂ and —CN, where each R″ is independently selected from H, —C₁-C₈ alkyl, —C₂-C₈ alkenyl, —C₂-C₈ alkynyl, or aryl wherein said further substituents are preferably unsubstituted. In some embodiments, the —O—(C₁-C₈ alkyl), —O—(C₂-C₈ alkenyl), —O—(C₂-C₈ alkynyl), aryl, and R′ groups are not further substituted.

Unless otherwise indicated by context, the terms “alkenyl” and “alkynyl” refer to substituted or unsubstituted straight and branched carbon chains having from 2 to 20 carbon atoms (and all combinations and subcombinations of ranges and specific numbers of carbon atoms therein), with from 2 to 3, 2 to 4, 2 to 8 or 2 to 10 carbon atoms being preferred. An alkenyl chain has at least one double bond in the chain and an alkynyl chain has at least one triple bond in the chain. Examples of alkenyl groups include, but are not limited to, ethylene or vinyl, allyl, -1 butenyl, -2 butenyl, -isobutylenyl, -1 pentenyl, -2 pentenyl, 3-methyl-1-butenyl, -2 methyl 2 butenyl, and -2,3 dimethyl 2 butenyl. Examples of alkynyl groups include, but are not limited to, acetylenic, propargyl, acetylenyl, propynyl, -1 butynyl, -2 butynyl, -1 pentynyl, -2 pentynyl, and -3 methyl 1 butynyl.

Alkenyl and alkynyl groups, whether alone or as part of another group, can be optionally substituted with one or more groups, preferably 1 to 3 groups (and any additional substituents selected from halogen), including but not limited to: halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —NH₂, —NH(R′), —N(R′)₂ and —CN; where each R′ is independently selected from H, —C₁-C₈ alkyl, —C₂-C alkenyl, —C₂-C₈ alkynyl, or aryl. In some embodiments, the —O—(C₁-C₈ alkyl), —O—(C₂-C₈ alkenyl), —O—(C₂-C₈ alkynyl), aryl, and R′ groups can be further substituted. Such further substituents include, for example, —C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —NH₂, —NH(R″), —N(R″)₂ and —CN, where each R″ is independently selected from H, —C₁-C₈ alkyl, —C₂-C₈ alkenyl, —C₂-C₈ alkynyl, or aryl, wherein said further substituents are preferably unsubstituted. In some embodiments, the —O—(C₁-C₈ alkyl), —O—(C₂-C₈ alkenyl), —O—(C₂-C₈ alkynyl), -aryl, and R′ groups are not further substituted.

Unless otherwise indicated by context, the term “alkylene” refers to a substituted or unsubstituted saturated branched or straight chain hydrocarbon radical having from 1 to 20 carbon atoms (and all combinations and subcombinations of ranges and specific numbers of carbon atoms therein), with from 1 to 8 or 1 to 10 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, octylene, nonylene, decalene, 1,4-cyclohexylene, and the like.

Alkylene groups, whether alone or as part of another group, can be optionally substituted with one or more groups, preferably 1 to 3 groups (and any additional substituents selected from halogen), including, but not limited to: halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)²R′, —S(O)R′, —OH, ═O, —NH₂, —NH(R′), —N(R′)₂ and —CN; where each R′ is independently selected from H, —C₁-C₈ alkyl, —C₂-C₈ alkenyl, —C₂-C₈ alkynyl, or -aryl. In some embodiments, the —O—(C₁-C₈ alkyl), —O—(C₂-C₈ alkenyl), —O—(C₂-C₈ alkynyl), aryl, and R′ groups can be further substituted. Such further substituents include, for example, C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —NH₂, —NH(R″), —N(R″)₂ and —CN, where each R″ is independently selected from H, —C₁-C₈ alkyl, —C₂-C₈ alkenyl, —C₂-C₈ alkynyl, or aryl wherein said further substituents are preferably unsubstituted. In some embodiments, the —O—(C₁-C₈ alkyl), —O—(C₂-C₈ alkenyl), —O—(C₂-C₈ alkynyl), -aryl, and R′ groups are not further substituted.

Unless otherwise indicated by context, the term “aryl” refers to a substituted or unsubstituted monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to: halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NO₂, —NH₂, —NH(R′), —N(R′)₂ and —CN; where each R′ is independently selected from H, —C₁-C₈ alkyl, —C₂-C₈ alkenyl, —C₂-C₈ alkynyl, or aryl. In some embodiments, the C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), aryl and R′ groups can be further substituted. Such further substituents include, for example, —C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —NH₂, —NH(R″), —N(R″)₂ and —CN, where each R″ is independently selected from —H, —C₁-C₈ alkyl, —C₂-C₈ alkenyl, —C₂-C₈ alkynyl, or aryl wherein said further substituents are preferably unsubstituted. In some embodiments, the —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), aryl and R′ groups are not further substituted.

Unless otherwise indicated by context, the term “heterocycle” refers to a substituted or unsubstituted monocyclic ring system having from 3 to 7, or 3 to 10, ring atoms (also referred to as ring members) wherein at least one ring atom is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocyclic heterocycle preferably has 3 to 7 ring members (e.g., 2 to 6 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure.

Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960).

Examples of “heterocycle” groups include by way of example and not limitation pyridyl, dihydropyridyl, tetrahydropyridyl (piperidyl), thiazolyl, pyrimidinyl, furanyl, thienyl, pyrrolyl, pyrazolyl, imidazolyl, tetrazolyl, fucosyl, aziridinyl, azetidinyl, oxiranyl, oxetanyl, and tetrahydrofuranyl.

A heterocycle group, whether alone or as part of another group, can be optionally substituted with one or more groups, preferably 1 to 2 groups, including but not limited to: —C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NH₂, —NH(R′), —N(R′)₂ and —CN; where each R′ is independently selected from H, —C₁-C₈ alkyl, —C₂-C₈ alkenyl, —C₂-C₈ alkynyl, or -aryl. In some embodiments, the O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, aryl, and R′ groups can be further substituted. Such further substituents include, for example, —C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —NH₂, —NH(R″), —N(R″)₂ and —CN, where each R″ is independently selected from H, —C₁-C₈ alkyl, —C₂-C₈ alkenyl, —C₂-C₈ alkynyl, or aryl wherein said further substituents are preferably unsubstituted. In some embodiments, the —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, aryl, and R′ groups are not substituted.

By way of example and not limitation, carbon-bonded heterocycles can be bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine. Exemplary carbon bonded heterocycles can include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles can be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole, or isoindoline; and position 4 of a morpholine. Still more typically, nitrogen bonded heterocycles include 1-aziridyl, 1-azetidyl, 1-pyrrolyl, 1-imidazolyl, 1-pyrazolyl, and 1-piperidinyl.

Unless otherwise noted, the term “carbocycle,” refers to a substituted or unsubstituted, saturated or unsaturated non-aromatic monocyclic ring system having from 3 to 6 ring atoms (and all combinations and subcombinations of ranges and specific numbers of carbon atoms therein) wherein all of the ring atoms are carbon atoms.

Carbocycle groups, whether alone or as part of another group, can be optionally substituted with, for example, one or more groups, preferably 1 or 2 groups (and any additional substituents selected from halogen), including, but not limited to: halogen, C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —NH₂, —NH(R′), —N(R′)₂ and —CN; where each R′ is independently selected from H, —C₁-C₈ alkyl, —C₂-C₈ alkenyl, —C₂-C₈ alkynyl, or aryl. In some embodiments, the —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl and R′ groups can be further substituted. Such further substituents include, for example, —C₁-C₈ alkyl, —C₂-C₈ alkenyl, —C₂-C₈ alkynyl, halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —NH₂, —NH(R″), —N(R″)₂ and —CN, where each R″ is independently selected from H, —C₁-C₈ alkyl, —C₂-C₈ alkenyl, —C₂-C₈ alkynyl, or aryl wherein said further substituents are preferably unsubstituted. In some embodiments, the —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), aryl and R′ groups are not substituted.

Examples of monocyclic carbocylic substituents include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, cyclooctyl, -1,3-cyclohexadienyl, -1,4-cyclohexadienyl, -1,3-cycloheptadienyl, -1,3,5-cycloheptatrienyl, and -cyclooctadienyl.

When any variable occurs more than one time in any constituent or in any formula, its definition in each occurrence is independent of its definition at every other. Combinations of substituents and/or variables are permissible only if such combinations result in stable compounds.

Unless otherwise indicated by context, a hyphen (-) designates the point of attachment to the pendant molecule. Accordingly, the term “—(C₁-C₁₀ alkylene)aryl” or “—C₁-C₁₀ alkylene(aryl)” refers to a C₁-C₁₀ alkylene radical as defined herein wherein the alkylene radical is attached to the pendant molecule at any of the carbon atoms of the alkylene radical and one of the hydrogen atom bonded to a carbon atom of the alkylene radical is replaced with an aryl radical as defined herein.

When a particular group is “substituted”, that group may have one or more substituents, preferably from one to five substituents, more preferably from one to three substituents, most preferably from one to two substituents, independently selected from the list of substituents. The group can, however, generally have any number of substituents selected from halogen.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

### Methods of Controlling Core Fucosylation

The invention provides methods for preparing antibodies and antibody derivatives with a specific level of core afucosylation. The methods are premised in part on the unexpected results presented in the Examples showing that accurate predictive models of antibody or antibody derivative afucosylation levels with a given fucosylation inhibitor (e.g., 2-fluorofucose) can be generated using parameters related to the fucosylation inhibitor (e.g., amount of fucosylation inhibitor or time of fucosylation inhibitor addition) paired with a corresponding culture parameter (e.g., integral of cell area or antibody titer) as inputs to the predictive model. As used herein, “core fucosylation” refers to addition of fucose (“fucosylation”) to N-acetylglucosamine (“GlcNAc”) at the reducing terminal of an N-linked glycan. Also provided are antibodies and antibody derivatives produced by such methods.

In some embodiments, the level of afucosylation of complex N-glycoside-linked sugar chains bound to the Fc region (or domain) of an antibody or antibody derivative is controlled by using a specific amount of a fucosylation inhibitor or adding the fucosylation inhibitor at a specific time during the antibody or antibody derivative culture. As used herein, a “complex N-glycoside-linked sugar chain” is typically bound to asparagine 297 (according to the number of Kabat), although a complex N-glycoside linked sugar chain can also be linked to other asparagine residues. As used herein, the complex N-glycoside-linked sugar chain has a biantennary composite sugar chain, mainly having the following structure:

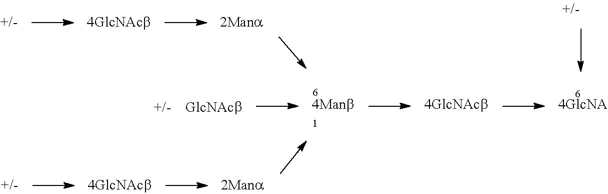

where ± indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” excludes a high mannose type of sugar chain, in which only mannose is incorporated at the non-reducing terminal of the core structure, but includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of the high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to the present methods, the amount of fucose incorporated into the complex N-glycoside-linked sugar chain(s) of an antibody or antibody derivative generated by culturing a cell line can be controlled. For example, in various embodiments, the antibody or antibody derivative has about any of 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5%, including any ranges between these values, core afucosylation. In some embodiments, the antibody or antibody derivative has greater than about any of 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5% core afucosylation.

Percent core fucosylation/afucosylation of an antibody or antibody derivative can be calculated using any method known in the art for carrying out such a determination. For example, Quadrupole Time of Flight (Qtof) mass spectrometer analysis can be used to calculate percent antibody core fucosylation/afucosylation. Qtof provides mass units and intensities of characteristic peaks of antibody heavy chains. In antibody core fucosylation Qtof analysis, spectra contain several peaks including: a peak that corresponds to the carbohydrate structure where there is no galactose at the two non-reducing termini and has core fucosylation (also referred to herein as “G0”); a peak that corresponds to a carbohydrate structure where one of the non-reducing termini has a galactose (a mixture of two isomers) and has core fucosylation (also referred to herein as “G1”); a peak that corresponds to a carbohydrate structure where both of the non-reducing termini have a galactose and has core fucosylation (also referred to herein as “G2”); a peak that corresponds to a carbohydrate structure where there is no galactose at either of the two non-reducing termini and there is no core fucosylation (also referred to herein as “G0-F”); a peak that corresponds to a carbohydrate structure where one of the non-reducing termini has a galactose (a mixture of two isomers) and there is no core fucosylation (also referred to herein as “G1-F”); and a peak that corresponds to a carbohydrate structure where both of the non-reducing termini have a galactose and there is no core fucosylation (also referred to herein as “G2-F”). The inhibition of fucosylation can be represented by a decrease of the intensity of the G0 peak and an increase of the G0-F peak. Thus, the percent afucosylation can be calculated using the following formula:

$$Percent Afucosylation = \frac{G 0 - F Peak Intensity}{G 0 - F Peak Intensity + G 0 Peak Intensity}$$

### Amount of Fucosylation Inhibitor

In some embodiments, the amount of fucose incorporated into the complex N-glycoside-linked sugar chain(s) of an antibody or antibody derivative generated by culturing a host cell can be controlled by varying the amount of a fucosylation inhibitor included in the culture medium. In some embodiments, the fucosylation inhibitor is a fucose analog. In some embodiments, the fucose analog is 2FF, the compound of formula I, or the compound of formula II. In some embodiments, the fucose analog is 2FF. In some embodiments, the amount of the fucosylation inhibitor added is less than a saturating amount that results in at least about 95% (such as at least about any of 96%, 97%, 98%, 99%, or greater) afucosylation when added at d0 of culturing. In some embodiments, the amount of the fucosylation inhibitor is determined using a predictive model that has concentration of the fucosylation inhibitor present in the culture medium and a culture parameter as inputs. In some embodiments, the culture parameter is integral cell area (ICA). In some embodiments, one or more (such as 2, 3, 4, 5, or more) additional amounts of the fucosylation inhibitor are added during culturing.

In some embodiments, the amount of fucose incorporated into the complex N-glycoside-linked sugar chain(s) of an antibody or antibody derivative generated by culturing a host cell can be controlled by varying the amount of a fucosylation inhibitor added at a specific time, Tₐ, during the culturing. In some embodiments, Tₐ is any of d0, d1, d2, d3, d4, d5, or later of the culturing. In some embodiments, Tₐ is no later than d5 (such as no later than any of d4, d3, d2, d1, or d0) of the culturing. In some embodiments, Tₐ is d0 of the culturing. In some embodiments, the fucosylation inhibitor is a fucose analog. In some embodiments, the fucose analog is 2FF, the compound of formula I, or the compound of formula II. In some embodiments, the fucose analog is 2FF. In some embodiments, the amount of the fucosylation inhibitor added is less than a saturating amount that results in at least about 95% (such as at least about any of 96%, 97%, 98%, 99%, or greater) afucosylation when added at d0. In some embodiments, the amount of the fucosylation inhibitor is determined using a predictive model that has concentration of the fucosylation inhibitor added at Tₐ and a culture parameter as inputs. In some embodiments, the culture parameter is integral cell area (ICA). In some embodiments, one or more (such as 2, 3, 4, 5, or more) additional amounts of the fucosylation inhibitor are added following Tₐ.

Thus, in some embodiments, provided herein is a method of controlling the level (e.g., percent) of afucosylation of an antibody or antibody derivative, comprising: (a) culturing a host cell in a culture medium in the presence of a pre-determined amount of an inhibitor of fucosylation (Aₚ), wherein the host cell expresses an antibody or antibody derivative having an Fc domain having at least one complex N-glycoside-linked sugar chain bound to the Fc domain through an N-acetylglucosamine of the reducing terminal of the sugar chain; and (b) isolating the antibody or antibody derivative. In some embodiments, the antibody or antibody derivative is isolated upon completion of culturing. In some embodiments, Aₚ is determined such that the level of afucosylation of the isolated antibody or antibody derivative of (b) has a level of afucosylation that does not exceed a maximum deviation from a target level of afucosylation. In some embodiments, the method further comprises determining Aₚ. In some embodiments, the fucosylation inhibitor is a fucose analog. In some embodiments, the fucose analog is 2FF, the compound of formula I, or the compound of formula II. In some embodiments, the fucose analog is 2FF. In some embodiments, Aₚ is less than a saturating amount that results in at least about 95% (such as at least about any of 96%, 97%, 98%, 99%, or greater) afucosylation when added at d0. In some embodiments, one or more (such as 2, 3, 4, 5, or more) additional amounts of the fucosylation inhibitor are added during culturing. In some embodiments, one or more of the additional amounts of the fucosylation inhibitor are, independently, the same or about the same as Aₚ. In some embodiments, one or more of the additional amounts of the fucosylation inhibitor are, independently, less than about Aₚ.

In some embodiments, provided herein is a method of controlling the level (e.g., percent) of afucosylation of an antibody or antibody derivative, comprising: (a) culturing a host cell in a culture medium, wherein the host cell expresses an antibody or antibody derivative having an Fc domain having at least one complex N-glycoside-linked sugar chain bound to the Fc domain through an N-acetylglucosamine of the reducing terminal of the sugar chain, and wherein a pre-determined amount of an inhibitor of fucosylation (Aₚ) is added to the culture medium at a time Tₐ; and (b) isolating the antibody or antibody derivative. In some embodiments, the antibody or antibody derivative is isolated upon completion of culturing. In some embodiments, Aₚ is determined such that the level of afucosylation of the isolated antibody or antibody derivative of (b) has a level of afucosylation that does not exceed a maximum deviation from a target level of afucosylation. In some embodiments, the method further comprises determining Aₚ. In some embodiments, the fucosylation inhibitor is a fucose analog. In some embodiments, the fucose analog is 2FF, the compound of formula I, or the compound of formula II. In some embodiments, the fucose analog is 2FF. In some embodiments, Aₚ is less than a saturating amount that results in at least about 95% (such as at least about any of 96%, 97%, 98%, 99%, or greater) afucosylation when added at d0. In some embodiments, Tₐ is any of d0, d1, d2, d3, d4, d5, or later of the culturing. In some embodiments, Tₐ is no later than d5 (such as no later than any of d4, d3, d2, d1, or d0) of the culturing. In some embodiments, Tₐ is d0 of the culturing. In some embodiments, one or more (such as 2, 3, 4, 5, or more) additional amounts of the fucosylation inhibitor are added following Tₐ. In some embodiments, one or more of the additional amounts of the fucosylation inhibitor are, independently, the same or about the same as Aₚ. In some embodiments, one or more of the additional amounts of the fucosylation inhibitor are, independently, less than about Aₚ.

In some embodiments, according to any of the methods described herein employing Aₚ, Aₚ is determined based on a predictive model generated using a plurality of different fucosylation inhibitor amounts (e.g., present in the culture medium from d0 or added at Tₐ) and a cell growth parameter of the host cell in the culture as inputs and the level of afucosylation of the isolated antibody or antibody derivative as the output. In some embodiments, the predictive model is generated using the fucosylation inhibitor amounts normalized to the cell growth parameter as inputs. In some embodiments, the method further comprises generating the predictive model. In some embodiments, the cell growth parameter is integral cell area (ICA).

In some embodiments, according to any of the methods described herein using a plurality of different fucosylation inhibitor amounts (e.g., present in the culture medium from d0 or added at Tₐ), at least 3 (such as at least 3, 4, 5, 6, 7, 8, 9, 10, or more) different amounts of the fucosylation inhibitor are used. In some embodiments, the plurality of different fucosylation inhibitor amounts spans a range of at least about a 10-fold (such as at least about any of a 25-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, 10,000-fold or more) difference in concentration. In some embodiments, the plurality of different fucosylation inhibitor amounts spans a range of at least about a 1,000-fold difference in concentration. In some embodiments, the plurality of different fucosylation inhibitor amounts does not include any amounts of the fucosylation inhibitor that exceed a saturating amount that results in at least about 95% (such as at least about any of 96%, 97%, 98%, 99%, or greater) afucosylation when added at d0. In some embodiments, the fucosylation inhibitor is a fucose analog. In some embodiments, the fucose analog is 2FF, the compound of formula I, or the compound of formula II. In some embodiments, the fucose analog is 2FF. In some embodiments, the fucosylation inhibitor is 2FF, and the plurality of different 2FF amounts does not include any concentrations greater than about 100 μM.

In some embodiments, according to any of the methods described herein employing Aₚ, Aₚ is less than a saturating amount that results in at least about 95% (such as at least about any of 96%, 97%, 98%, 99%, or greater) afucosylation when added at d0. In some embodiments, Aₚ is about any of 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 120 μM, 140 μM, 160 μM, 180 μM, 200 μM, 220 μM, 240 μM, 260 μM, 280 μM, 300 μM, 320 μM, 340 μM, 360 μM, 380 μM, 400 μM, 420 μM, 440 μM, 460 μM, 480 μM, 500 μM, 520 μM, 540 μM, 560 μM, 580 μM, 600 μM, 620 μM, 640 μM, 660 μM, 680 μM, 700 μM, 720 μM, 740 μM, 760 μM, 780 μM, 800 μM, 820 μM, 840 μM, 860 μM, 880 μM, 900 μM, 920 μM, 940 μM, 960 μM, 980 μM, 1,000 μM, or more, including any ranges between these values. In some embodiments, the fucosylation inhibitor is a fucose analog. In some embodiments, the fucose analog is 2FF, the compound of formula I, or the compound of formula II. In some embodiments, the fucose analog is 2FF.

In some embodiments, according to any of the methods described herein employing Aₚ, the fucosylation inhibitor is 2FF, and Aₚ is less than about 100 μM (such as less than about any of 99 μM, 98 μM, 97 μM, 96 μM, 95 μM, 94 μM, 93 μM, 92 μM, 91 μM, 90 μM, 89 μM, 88 μM, 87 μM, 86 μM, 85 μM, 84 μM, 83 μM, 82 μM, 81 μM, 80 μM, 79 μM, 78 μM, 77 μM, 76 μM, 75 μM, 74 μM, 73 μM, 72 μM, 71 μM, 70 μM, 69 μM, 68 μM, 67 μM, 66 μM, 65 μM, 64 μM, 63 μM, 62 μM, 61 μM, 60 μM, 59 μM, 58 μM, 57 μM, 56 μM, 55 μM, 54 μM, 53 μM, 52 μM, 51 μM, 50 μM, 49 μM, 48 μM, 47 μM, 46 μM, 45 μM, 44 μM, 43 μM, 42 μM, 41 μM, 40 μM, 39 μM, 38 μM, 37 μM, 36 μM, 35 μM, 34 μM, 33 μM, 32 μM, 31 μM, 30 μM, 29 μM, 28 μM, 27 μM, 26 μM, 25 μM, 24 μM, 23 μM, 22 μM, 21 μM, 20 μM, 19 μM, 18 μM, 17 μM, 16 μM, 15 μM, 141 μM, 131 μM, 121 μM, 11 μM, 10 μM, 9 μM, 8 μM, 7 μM, 6 μM, 5 μM, 4 μM, 3 μM, 2 μM, or 1 μM).

In some embodiments, the predictive model is a statistical model generated by plotting % afucosylation against 2FF concentration present in the culture medium normalized to ICA and fitting the curve to a Michaelis-Menten kinetics equation to determine the constants in the equation.

In some embodiments, according to any of the methods described herein employing Aₚ and Tₐ, Tₐ is any of d0, d1, d2, d3, d4, d5, or later of the culturing. In some embodiments, Tₐ is no later than d5 (such as no later than any of d4, d3, d2, d1, or d0) of the culturing. In some embodiments, Tₐ is d0 of the culturing.

In some embodiments, according to any of the methods described herein employing Aₚ, the maximum deviation from the target level of afucosylation is no more than about any of 10%, 9%, 8%, 7%, 6%, 5%, 4%, 3%, 2%, 1%, or less. In some embodiments, the maximum deviation from the target level of afucosylation is no more than about 5%.

In some embodiments, according to any of the methods described herein employing Aₚ, the antibody or antibody derivative has about any of 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5%, including any ranges between these values, core afucosylation. In some embodiments, the antibody or antibody derivative has greater than about any of 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5% core afucosylation. In some embodiments, the antibody or antibody derivative has about 100% to about 95% core afucosylation. In some embodiments, the antibody or antibody derivative has about 95% to about 90% core afucosylation. In some embodiments, the antibody or antibody derivative has about 90% to about 85% core afucosylation. In some embodiments, the antibody or antibody derivative has about 85% to about 80% core afucosylation. In some embodiments, the antibody or antibody derivative has about 80% to about 75% core afucosylation. In some embodiments, the antibody or antibody derivative has about 75% to about 70% core afucosylation. In some embodiments, the antibody or antibody derivative has about 70% to about 65% core afucosylation. In some embodiments, the antibody or antibody derivative has about 65% to about 60% core afucosylation. In some embodiments, the antibody or antibody derivative has about 60% to about 55% core afucosylation. In some embodiments, the antibody or antibody derivative has about 55% to about 50% core afucosylation. In some embodiments, the antibody or antibody derivative has about 50% to about 45% core afucosylation. In some embodiments, the antibody or antibody derivative has about 45% to about 40% core afucosylation. In some embodiments, the antibody or antibody derivative has about 40% to about 35% core afucosylation. In some embodiments, the antibody or antibody derivative has about 35% to about 30% core afucosylation. In some embodiments, the antibody or antibody derivative has about 30% to about 25% core afucosylation. In some embodiments, the antibody or antibody derivative has about 25% to about 20% core afucosylation. In some embodiments, the antibody or antibody derivative has about 20% to about 15% core afucosylation. In some embodiments, the antibody or antibody derivative has about 15% to about 10% core afucosylation. In some embodiments, the antibody or antibody derivative has about 10% to about 5% core afucosylation. In some embodiments, the antibody or antibody derivative has about 5% to about 0% core afucosylation.

In some embodiments, provided herein is a method of controlling the level (e.g., percent) of afucosylation of an antibody or antibody derivative, comprising: (a) culturing a host cell in a culture medium comprising a pre-determined amount of 2FF (Aₚ), wherein the host cell expresses an antibody or antibody derivative having an Fc domain having at least one complex N-glycoside-linked sugar chain bound to the Fc domain through an N-acetylglucosamine of the reducing terminal of the sugar chain; and (b) isolating the antibody or antibody derivative. In some embodiments, the antibody or antibody derivative is isolated upon completion of culturing. In some embodiments, Aₚ is determined such that the level of afucosylation of the isolated antibody or antibody derivative of (b) has a level of afucosylation that does not exceed a maximum deviation from a target level of afucosylation. In some embodiments, the method further comprises determining Aₚ. In some embodiments, Aₚ is less than a saturating amount that results in at least about 95% (such as at least about any of 96%, 97%, 98%, 99%, or greater) afucosylation when added at d0. In some embodiments, Aₚ is less than about 100 μM (such as less than about any of 90 μM, 80 μM, 70 μM, 60 μM, 50 μM, 40 μM, 30 μM, 20 μM, 10 μM, 5 μM, or less).

In some embodiments, provided herein is a method of controlling the level (e.g., percent) of afucosylation of an antibody or antibody derivative, comprising: (a) culturing a host cell in a culture medium comprising a pre-determined amount of 2FF (Aₚ), wherein the host cell expresses an antibody or antibody derivative having an Fc domain having at least one complex N-glycoside-linked sugar chain bound to the Fc domain through an N-acetylglucosamine of the reducing terminal of the sugar chain; and (b) isolating the antibody or antibody derivative, wherein Aₚ is determined based on a predictive model generated using a plurality of different 2FF amounts added at Tₐ and a cell growth parameter of the host cell in the culture as inputs and the level of afucosylation of the antibody or antibody derivative isolated as the output. In some embodiments, the antibody or antibody derivative is isolated upon completion of culturing. In some embodiments, the predictive model is generated using the 2FF amounts normalized to the cell growth parameter as inputs. In some embodiments, the method further comprises generating the predictive model. In some embodiments, the cell growth parameter is integral cell area (ICA). In some embodiments, Aₚ is determined such that the level of afucosylation of the isolated antibody or antibody derivative of (b) has a level of afucosylation that does not exceed a maximum deviation from a target level of afucosylation. In some embodiments, the method further comprises determining Aₚ. In some embodiments, Aₚ is less than a saturating amount that results in at least about 95% (such as at least about any of 96%, 97%, 98%, 99%, or greater) afucosylation when added at d0. In some embodiments, Aₚ is less than about 100 μM (such as less than about any of 90 μM, 80 μM, 70 μM, 60 μM, 50 μM, 40 μM, 30 μM, 20 μM, 10 μM, 5 μM, or less).

### Time of Fucosylation Inhibitor Addition

In some embodiments, the amount of fucose incorporated into the complex N-glycoside-linked sugar chain(s) of an antibody or antibody derivative generated by culturing a host cell can be controlled by varying the time during the culturing at which a fucosylation inhibitor is added. In some embodiments, the fucosylation inhibitor is added following d0 of the culturing. In some embodiments, the fucosylation inhibitor is a fucose analog. In some embodiments, the fucose analog is 2-fluorofucose (2FF), the compound of formula I, or the compound of formula II. In some embodiments, the fucose analog is 2FF. In some embodiments, the amount of the fucosylation inhibitor added is at or about a saturating amount that results in at least about 95% (such as at least about any of 96%, 97%, 98%, 99%, or greater) afucosylation when added at d0. In some embodiments, the amount of the fucosylation inhibitor added is less than about a saturating amount that results in at least about 95% (such as at least about any of 96%, 97%, 98%, 99%, or greater) afucosylation when added at d0. In some embodiments, the time of addition of the fucosylation inhibitor is determined using a predictive model that has fucosylation inhibitor addition time and a culture parameter as inputs. In some embodiments, the culture parameter is antibody titer. In some embodiments, the antibody titer is the antibody titer at the time of addition of the fucosylation inhibitor. In some embodiments, one or more (such as 2, 3, 4, 5, or more) additional amounts of the fucosylation inhibitor are added following the first addition.

Thus, in some embodiments, provided herein is a method of controlling the level of afucosylation of an antibody or antibody derivative, comprising: (a) culturing a host cell in a culture medium, wherein the host cell expresses an antibody or antibody derivative having an Fc domain having at least one complex N-glycoside-linked sugar chain bound to the Fc domain through an N-acetylglucosamine of the reducing terminal of the sugar chain; (b) adding an amount of an inhibitor of fucosylation to the culture medium at a pre-determined time (Tₚ) during the culturing; and (c) isolating the antibody or antibody derivative. In some embodiments, the antibody or antibody derivative is isolated upon completion of culturing. In some embodiments, Tₚ is determined such that the level of afucosylation of the isolated antibody or antibody derivative of (c) has a level of afucosylation that does not exceed a maximum deviation from a target level of afucosylation. In some embodiments, the method further comprises determining Tₚ. In some embodiments, the fucosylation inhibitor is a fucose analog. In some embodiments, the fucose analog is 2FF, the compound of formula I, or the compound of formula II. In some embodiments, the fucose analog is 2FF. In some embodiments, the amount of the fucosylation inhibitor added is at or about a saturating amount that results in at least about 95% (such as at least about any of 96%, 97%, 98%, 99%, or greater) afucosylation when added at d0. In some embodiments, the amount of the fucosylation inhibitor added is less than about a saturating amount that results in at least about 95% (such as at least about any of 96%, 97%, 98%, 99%, or greater) afucosylation when added at d0. In some embodiments, one or more (such as 2, 3, 4, 5, or more) additional amounts of the fucosylation inhibitor are added following the first addition. In some embodiments, one or more of the additional amounts of the fucosylation inhibitor are, independently, the same or about the same as the amount of the fucosylation inhibitor in the first addition. In some embodiments, one or more of the additional amounts of the fucosylation inhibitor are, independently, less than about the amount of the fucosylation inhibitor in the first addition.

In some embodiments, according to any of the methods described herein employing Tₚ, Tₚ is determined based on a predictive model generated using titer of the antibody or antibody derivative in the culture at a plurality of different fucosylation inhibitor addition times in the culturing as inputs and the level of afucosylation of the antibody or antibody derivative isolated as the output. In some embodiments, the method further comprises generating the predictive model.

In some embodiments, according to any of the methods described herein using a plurality of different fucosylation inhibitor addition times, at least 3 (such as at least 3, 4, 5, 6, 7, 8, 9, 10, or more) different addition times of the fucosylation inhibitor are used. In some embodiments, the plurality of different fucosylation inhibitor addition times spans a range of at least about a 24 hours (such as at least about any of 36 hours, 48 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or more). In some embodiments, the plurality of different fucosylation inhibitor addition times spans a range of at least about 72 hours. In some embodiments, the fucosylation inhibitor is a fucose analog. In some embodiments, the fucose analog is 2FF, the compound of formula I, or the compound of formula II. In some embodiments, the fucose analog is 2FF.

In some embodiments, according to any of the methods described herein employing Tₚ, the amount of the fucosylation inhibitor added at Tₚ is about any of 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 120 μM, 140 μM, 160 μM, 180 μM, 200 μM, 220 μM, 240 μM, 260 μM, 280 μM, 300 μM, 320 μM, 340 μM, 360 μM, 380 μM, 400 μM, 420 μM, 440 μM, 460 μM, 480 μM, 500 μM, 520 μM, 540 μM, 560 μM, 580 μM, 600 μM, 620 μM, 640 μM, 660 μM, 680 μM, 700 μM, 720 μM, 740 μM, 760 μM, 780 μM, 800 μM, 820 μM, 840 μM, 860 μM, 880 μM, 900 μM, 920 μM, 940 μM, 960 μM, 980 μM, 1,000 μM, or more, including any ranges between these values. In some embodiments, the fucosylation inhibitor is a fucose analog. In some embodiments, the fucose analog is 2FF, the compound of formula I, or the compound of formula II. In some embodiments, the fucose analog is 2FF.

In some embodiments, according to any of the methods described herein employing Tₚ, the fucosylation inhibitor is 2FF, and the amount of 2FF added at Tₚ is about or less than about 100 μM (such as less than about any of 99 μM, 98 μM, 97 μM, 96 μM, 95 μM, 94 μM, 93 μM, 92 μM, 91 μM, 90 μM, 89 μM, 88 μM, 87 μM, 86 μM, 85 μM, 84 μM, 83 μM, 82 μM, 81 μM, 80 μM, 79 μM, 78 μM, 77 μM, 76 μM, 75 μM, 74 μM, 73 μM, 72 μM, 71 μM, 70 μM, 69 μM, 68 μM, 67 μM, 66 μM, 65 μM, 64 μM, 63 μM, 62 μM, 61 μM, 60 μM, 59 μM, 58 μM, 57 μM, 56 μM, 55 μM, 54 μM, 53 μM, 52 μM, 51 μM, 50 μM, 49 μM, 48 μM, 47 μM, 46 μM, 45 μM, 44 μM, 43 μM, 42 μM, 41 μM, 40 μM, 39 μM, 38 μM, 37 μM, 36 μM, 35 μM, 34 μM, 33 μM, 32 μM, 31 μM, 30 μM, 29 μM, 28 μM, 27 μM, 26 μM, 25 μM, 24 μM, 23 μM, 22 μM, 21 μM, 20 μM, 19 μM, 18 μM, 17 μM, 16 μM, 15 μM, 14 μM, 13 μM, 12 μM, 11 μM, 10 μM, 9 μM, 8 μM, 7 μM, 6 μM, 5 μM, 4 μM, 3 μM, 21 μM, or 1 μM).

In some embodiments, according to any of the methods described herein employing Tₚ, the maximum deviation from the target level of afucosylation is no more than about any of 10%, 9%, 8%, 7%, 6%, 5%, 4%, 3%, 2%, 1%, or less. In some embodiments, the maximum deviation from the target level of afucosylation is no more than about 5%.

In some embodiments, according to any of the methods described herein employing Tₚ, the antibody or antibody derivative has about any of 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5%, including any ranges between these values, core afucosylation. In some embodiments, the antibody or antibody derivative has greater than about any of 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5% core afucosylation. In some embodiments, the antibody or antibody derivative has about 100% to about 95% core afucosylation. In some embodiments, the antibody or antibody derivative has about 95% to about 90% core afucosylation. In some embodiments, the antibody or antibody derivative has about 90% to about 85% core afucosylation. In some embodiments, the antibody or antibody derivative has about 85% to about 80% core afucosylation. In some embodiments, the antibody or antibody derivative has about 80% to about 75% core afucosylation. In some embodiments, the antibody or antibody derivative has about 75% to about 70% core afucosylation. In some embodiments, the antibody or antibody derivative has about 70% to about 65% core afucosylation. In some embodiments, the antibody or antibody derivative has about 65% to about 60% core afucosylation. In some embodiments, the antibody or antibody derivative has about 60% to about 55% core afucosylation. In some embodiments, the antibody or antibody derivative has about 55% to about 50% core afucosylation. In some embodiments, the antibody or antibody derivative has about 50% to about 45% core afucosylation. In some embodiments, the antibody or antibody derivative has about 45% to about 40% core afucosylation. In some embodiments, the antibody or antibody derivative has about 40% to about 35% core afucosylation. In some embodiments, the antibody or antibody derivative has about 35% to about 30% core afucosylation. In some embodiments, the antibody or antibody derivative has about 30% to about 25% core afucosylation. In some embodiments, the antibody or antibody derivative has about 25% to about 20% core afucosylation. In some embodiments, the antibody or antibody derivative has about 20% to about 15% core afucosylation. In some embodiments, the antibody or antibody derivative has about 15% to about 10% core afucosylation. In some embodiments, the antibody or antibody derivative has about 10% to about 5% core afucosylation. In some embodiments, the antibody or antibody derivative has about 5% to about 0% core afucosylation.

In some embodiments, provided herein is a method of controlling the level of afucosylation of an antibody or antibody derivative, comprising: (a) culturing a host cell in a culture medium, wherein the host cell expresses an antibody or antibody derivative having an Fc domain having at least one complex N-glycoside-linked sugar chain bound to the Fc domain through an N-acetylglucosamine of the reducing terminal of the sugar chain; (b) adding 2FF to the culture medium at a pre-determined time (Tₚ) during the culturing; and (c) isolating the antibody or antibody derivative. In some embodiments, the antibody or antibody derivative is isolated upon completion of culturing. In some embodiments, Tₚ is determined such that the level of afucosylation of the isolated antibody or antibody derivative of (c) has a level of afucosylation that does not exceed a maximum deviation from a target level of afucosylation. In some embodiments, the method further comprises determining Tₚ. In some embodiments, the amount of 2FF added is at or about a saturating amount that results in at least about 95% (such as at least about any of 96%, 97%, 98%, 99%, or greater) afucosylation when added at d0. In some embodiments, the amount of 2FF added is about 100 μM.

In some embodiments, provided herein is a method of controlling the level of afucosylation of an antibody or antibody derivative, comprising: (a) culturing a host cell in a culture medium, wherein the host cell expresses an antibody or antibody derivative having an Fc domain having at least one complex N-glycoside-linked sugar chain bound to the Fc domain through an N-acetylglucosamine of the reducing terminal of the sugar chain; (b) adding 2FF to the culture medium at a pre-determined time (Tₚ) during the culturing; and (c) isolating the antibody or antibody derivative, wherein Tₚ is determined based on a predictive model generated using titer of the antibody or antibody derivative in the culture at a plurality of different 2FF addition times in the culturing as inputs and the level of afucosylation of the antibody or antibody derivative isolated as the output. In some embodiments, the antibody or antibody derivative is isolated upon completion of culturing. In some embodiments, the method further comprises generating the predictive model. In some embodiments, Tₚ is determined such that the level of afucosylation of the isolated antibody or antibody derivative of (c) has a level of afucosylation that does not exceed a maximum deviation from a target level of afucosylation. In some embodiments, the method further comprises determining Tₚ. In some embodiments, the amount of 2FF added is at or about a saturating amount that results in at least about 95% (such as at least about any of 96%, 97%, 98%, 99%, or greater) afucosylation when added at d0. In some embodiments, the amount of 2FF added is about 100 μM.

In some embodiments, according to any of the methods of controlling the level of afucosylation of an antibody or antibody derivative described herein, the host cell is a recombinant host cell. In some embodiments, the host cell is a Chinese hamster ovary (CHO) cell.

In some embodiments, according to any of the methods of controlling the level of afucosylation of an antibody or antibody derivative described herein, the host cell is a hybridoma.

In some embodiments, according to any of the methods of controlling the level of afucosylation of an antibody or antibody derivative described herein, the host cell is grown in fed batch culture.

In some embodiments, according to any of the methods of controlling the level of afucosylation of an antibody or antibody derivative described herein, the host cell is grown in continuous feed culture.

In some embodiments, according to any of the methods of controlling the level of afucosylation of an antibody or antibody derivative described herein, the culture medium has a volume of at least 100 liters. In some embodiments, the culture medium has a volume of at least 500 liters.

In some embodiments, according to any of the methods of controlling the level of afucosylation of an antibody or antibody derivative described herein, the culture media is an animal protein free media.

In some embodiments, according to any of the methods of controlling the level of afucosylation of an antibody or antibody derivative described herein, isolating the antibody or antibody derivative comprises isolating the antibody or antibody derivative from the cell and/or the culture medium. In some embodiments, isolating the antibody or antibody derivative comprises using a protein A column. In some embodiments, isolating the antibody or antibody derivative comprises using a cation or anion exchange column or a hydrophobic interaction column.

In some embodiments, according to any of the methods of controlling the level of afucosylation of an antibody or antibody derivative described herein, the antibody or antibody derivative is an intact antibody. In some embodiments, the intact antibody is an IgG1 antibody.

In some embodiments, according to any of the methods of controlling the level of afucosylation of an antibody or antibody derivative described herein, the antibody or antibody derivative is a single chain antibody.

In some embodiments, according to any of the methods of controlling the level of afucosylation of an antibody or antibody derivative described herein, the antibody or antibody derivative comprises a heavy chain variable region, a light chain variable region, and an Fc region.

In some embodiments, according to any of the methods of controlling the level of afucosylation of an antibody or antibody derivative described herein, the antibody or antibody derivative is an antibody derivative comprising an antibody Fc region and a ligand binding domain of a non-immunoglobulin protein.

### Fucosylation Inhibitors

In some embodiments, the methods described herein employ a fucosylation inhibitor. In some embodiments, the fucosylation inhibitor is 2-fluorofucose (2FF) or a fucose analog that, when administered to a subject, is converted in vivo to 2FF. Additional fucosylation inhibitors contemplated include those disclosed in U.S. Pat. No. 8,163,551 and U.S. Patent Publication No. 20150238509, which are incorporated herein by reference in their entireties. For example, in some embodiments, the fucosylation inhibitor is the fucose analog of formula I or II identified below.

In some embodiments, the fucose analog has the following formula (I) or (II):

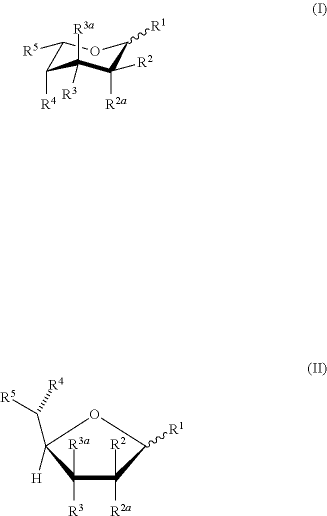

or a biologically acceptable salt or solvate thereof, wherein each of formula (I) or (II) can be the alpha or beta anomer or the corresponding aldose form;
each of R¹, R², R^{2a}, R³, R^{3a} and R⁴ is independently selected from OH, a hydrolyzable ester group, a hydrolyzable ether group, and a small electron withdrawing group;

R⁵ is a member selected from the group consisting of —CH₃, —CHX₂, —CH₂X, —CH(X′)—C₁-C₄ alkyl unsubstituted or substituted with halogen, —CH(X′)—C₂-C₄ alkene unsubstituted or substituted with halogen, —CH(X′)—C₂-C₄ alkyne unsubstituted or substituted with halogen, —CH═C(R¹⁰)(R¹¹), —C(CH₃)═C(R¹²)(R¹³), —C(R¹⁴)═C═C(R¹⁵)(R¹⁶), —C₃ carbocycle unsubstituted or substituted with methyl or halogen, —CH(X′)—C₃ carbocycle unsubstituted or substituted with methyl or halogen, C₃ heterocyle unsubstituted or substituted with methyl or halogen, —CH(X′)—C₃ heterocycle unsubstituted or substituted with methyl or halogen, —CH₂N₃, —CH₂CH₂N₃, and benzyloxymethyl, or R⁵ is a small electron withdrawing group; wherein

R¹⁰ is hydrogen or C₁-C₃ alkyl unsubstituted or substituted with halogen;
R¹¹ is C₁-C₃ alkyl unsubstituted or substituted with halogen;
R¹² is hydrogen, halogen or C₁-C₃ alkyl unsubstituted or substituted with halogen; and
R¹³ is hydrogen, or C₁-C₃ alkyl unsubstituted or substituted with halogen;
R¹⁴ is hydrogen or methyl;
R¹⁵ and R¹⁶ are independently selected from hydrogen, methyl and halogen;
X is halogen; and
X′ is halogen or hydrogen; and
additionally, each of R¹, R², R^{2a}, R³ and R^{3a} are optionally hydrogen; optionally two R¹, R², R^{2a}, R³ and R^{3a} on adjacent carbon atoms are combined to form a double bond between said adjacent carbon atoms; and
provided that at least one of R¹, R², R^{2a}, R³, R^{3a}, R⁴ and R⁵ is a small electron withdrawing group, or R⁵ comprises a halogen, site of unsaturation, carbocycle, heterocycle or azide.

In some selected embodiments, the fucose analog has the formula:

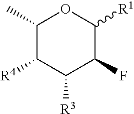

or an aldose form thereof, wherein each of R¹, R³, and R⁴ is independently —OH or a hydrolyzable ester group. In some embodiments, the hydrolyzable ester group is —OC(O)C₁-C₁₀ alkyl. In some selected embodiments, the hydrolyzable ester group is —OC(O)CH₃. In some embodiments, each of R¹, R³ and R⁴ is independently selected from the group consisting of —OH and —OC(O)C₁-C₁₀ alkyl. In some embodiments, each of R¹, R³ and R⁴ is independently selected from the group consisting of —OH and —OC(O)CH₃. In some embodiments, each of R¹, R³ and R⁴ is —OH. In some selected embodiments, the fucose analog is 2-deoxy-2-fluoro-L-fucose.

### Antibodies and Antibody Derivatives

Antibodies that can be produced by the instant methods can be monoclonal, chimeric, humanized (including veneered), or human antibodies. Suitable antibodies also include antibody fragments, such as single chain antibodies, or the like that have a Fc region or domain having a complex N-glycoside-linked sugar chain (e.g., a human IgG1 Fc region or domain). The Fc region or domain can include an Fcgamma receptor binding site. Typically, the antibodies are human or humanized. In some embodiments, the antibodies can be rodent (e.g., mouse and rat), donkey, sheep, rabbit, goat, guinea pig, camelid, horse, or chicken.

The antibodies can be mono-specific, bi-specific, tri-specific, or of greater multi-specificity. Multi-specific antibodies may be specific for different epitopes of different target antigens or may be specific for different epitopes on the same target antigen. (See, e.g., WO 93/17715; WO 92/08802; WO 91/00360; WO 92/05793; Tutt et al., 1991, J. Immunol. 147:60-69; U.S. Pat. Nos. 4,474,893; 4,714,681; 4,925,648; 5,573,920; and U.S. Pat. No. 5,601,819; Kostelny et al., 1992, J. Immunol. 148:1547-1553.)

The antibodies can also be described in terms of their binding affinity to a target antigen of 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments, the antibody is a chimeric antibody. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. (See, e.g., Morrison, Science, 1985, 229:1202; Oi et al., 1986, BioTechniques 4:214; Gillies et al., 1989, J. Immunol. Methods 125:191-202; U.S. Pat. Nos. 5,807,715; 4,816,567; and 4,816,397.)

In some embodiments, the antibody can be a humanized antibody, including a veneered antibody. Humanized antibodies are antibody molecules that bind the desired antigen and have one or more complementarity determining regions (CDRs) from a non-human species, and framework and constant regions from a human immunoglobulin molecule. Often, framework residues in the human framework regions will be substituted with the corresponding residue from the CDR donor antibody to alter, or preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riecbmann et al., 1988, Nature 332:323.) Antibodies can be humanized using a variety of techniques known in the art such as CDR-grafting (EP 0 239 400; WO 91/09967; U.S. Pat. Nos. 5,225,539; 5,530,101; and 5,585,089), veneering or resurfacing (EP 0 592 106; EP 0 519 596; Padlan, 1991, Molecular Immunology, 28(4/5):489-498; Studnicka et al., 1994, Protein Engineering 7(6):805-814; Roguska et al., 1994, Proc. Natl. Acad. Sci. USA 91:969-973), and chain shuffling (U.S. Pat. No. 5,565,332) (all of these references are incorporated by reference herein).

The antibody can also be a human antibody. Human antibodies can be made by a variety of methods known in the art such as phage display methods using antibody libraries derived from human immunoglobulin sequences. See e.g., U.S. Pat. Nos. 4,444,887 and 4,716,111; WO 98/46645, WO 98/50433, WO 98/24893, WO 98/16654, WO 96/34096, WO 96/33735, and WO 91/10741. In addition, a human antibody recognizing a selected epitope can be generated using a technique referred to as “guided selection,” in which a selected non-human monoclonal antibody, e.g., a mouse antibody, is used to guide the selection of a completely human antibody recognizing the same epitope (see, e.g., Jespers et al., 1994, Biotechnology 12:899-903). Human antibodies can also be produced using transgenic mice that express human immunoglobulin genes. Monoclonal antibodies directed against the antigen can be obtained from the immunized, transgenic mice using conventional hybridoma technology. For an overview of the technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598, 877; U.S. Pat. Nos. 5,413,923; 5,625,126; 5,633,425; 5,569,825; 5,661,016; 5,545,806; 5,814,318; 5,885,793; 5,916,771; and 5,939,598.

Examples of antibodies include HERCEPTIN® (trastuzumab; Genentech), RITUXAN® (rituximab; Genentech), lintuzumab (Seattle Genetics, Inc.), Palivizumab (Medimmune), Alemtuzumab (BTG) and Epratuzumab (Immunomedics).

In exemplary embodiments, an antibody or antibody derivative specifically binds to CD19, CD20, CD21, CD22, CD30, CD33, CD38, CD40, CD70, CD133, CD138, or CD276. In other embodiments, the antibody or antibody derivative specifically binds to BMPR1B, LAT1 (SLC7A5), STEAP1, MUC16, megakaryocyte potentiating factor (MPF), Napi3b, Sema 5b, PSCA hlg, ETBR (Endothelin type B receptor), STEAP2, TrpM4, CRIPTO, CD21, CD79a, CD79b, FcRH2, HER2, HER3, HER4, NCA, MDP, IL20Rα, Brevican, Ephb2R, ASLG659, PSCA, PSMA, GEDA, BAFF-R, CXCR5, HLA-DOB, P2X5, CD72, LY64, FCRH1, or IRTA2.

Antibodies can be assayed for specific binding to a target antigen by conventional methods, such as for example, competitive and non-competitive immunoassay systems using techniques such as Western blots, radioimmunoassays, ELISA (enzyme linked immunosorbent assay), “sandwich” immunoassays, immunoprecipitation assays, immunoradiometric assays, fluorescent immunoassays, and protein A immunoassays. (See, e.g., Ausubel et al., eds., Short Protocols in Molecular Biology (John Wiley & Sons, Inc., New York, 4th ed. 1999); Harlow & Lane, Using Antibodies: A Laboratory Manual (Cold Spring Harbor Laboratory Press, Cold Spring Harbor, N.Y., 1999.)

Further, the binding affinity of an antibody to a target antigen and the off-rate of an antibody-antigen interaction can be determined by surface plasmon resonance, competition FACS using labeled antibodies or other competitive binding assays. One example of a competitive binding assay is a radioimmunoassay comprising the incubation of labeled antigen (e.g., ³H or ¹²⁵I) with the antibody of interest in the presence of increasing amounts of unlabeled antibody, and the detection of the antibody bound to the labeled antigen. The affinity of the antibody and the binding off-rates can then be determined from the data by Scatchard plot analysis. Competition with a second antibody can also be determined using radioimmunoassays. In this case, the antigen is incubated with the antibody of interest conjugated to a labeled compound (e.g., ³H or ¹²⁵I) in the presence of increasing amounts of an unlabeled second antibody. Alternatively, the binding affinity of an antibody and the on- and off-rates of an antibody-antigen interaction can be determined by surface plasmon resonance.

Antibodies can be made from antigen-containing fragments of the target antigen by standard procedures according to the type of antibody (see, e.g., Kohler, et al., Nature, 256:495, (1975); Harlow & Lane, Antibodies, A Laboratory Manual (C. S. H. P., NY, 1988); Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes). As an example, monoclonal antibodies can be prepared using a wide variety of techniques including, e.g., the use of hybridoma, recombinant, and phage display technologies, or a combination thereof. Hybridoma techniques are generally discussed in, e.g., Harlow et al., supra, and Hammerling, et al., In Monoclonal Antibodies and T-Cell Hybridomas, pp. 563-681 (Elsevier, N.Y., 1981). Examples of phage display methods that can be used to make antibodies include, e.g., those disclosed in Briinnan et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01 134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108 (the disclosures of which are incorporated by reference herein).

Examples of techniques that can be used to produce single-chain Fvs and antibodies include those described in U.S. Pat. Nos. 4,946,778 and 5,258,498; Huston et al., 1991, Methods in Enzymology 203:46-88; Shu et al., 1993, Proc. Natl. Acad. Sci. USA 90:7995-7999; and Skerra et al., 1988, Science 240:1038-1040.

Examples of antibody derivatives include binding domain-Ig fusions, wherein the binding domain may be, for example, a ligand, an extracellular domain of a receptor, a peptide, a non-naturally occurring peptide or the like. Exemplary fusions with immunoglobulin or Fc regions include: etanercept which is a fusion protein of sTNFRII with the Fc region (U.S. Pat. No. 5,605,690), alefacept which is a fusion protein of LFA-3 expressed on antigen presenting cells with the Fc region (U.S. Pat. No. 5,914,111), a fusion protein of Cytotoxic T Lymphocyte-associated antigen-4 (CTLA-4) with the Fc region (J. Exp. Med. 181:1869 (1995)), a fusion protein of interleukin 15 with the Fc region (J. Immunol. 160:5742 (1998)), a fusion protein of factor VII with the Fc region (Proc. Natl. Acad. Sci. USA 98:12180 (2001)), a fusion protein of interleukin 10 with the Fc region (J. Immunol. 154:5590 (1995)), a fusion protein of interleukin 2 with the Fc region (J. Immunol. 146:915 (1991)), a fusion protein of CD40 with the Fc region (Surgery 132:149 (2002)), a fusion protein of Flt-3 (fms-like tyrosine kinase) with the antibody Fc region (Acta. Haemato. 95:218 (1996)), a fusion protein of OX40 with the antibody Fc region (J. Leu. Biol. 72:522 (2002)), and fusion proteins with other CD molecules (e.g., CD2, CD30 (TNFRSF8), CD95 (Fas), CD106 (VCAM-I), CD137), adhesion molecules (e.g., ALCAM (activated leukocyte cell adhesion molecule), cadherins, ICAM (intercellular adhesion molecule)-1, ICAM-2, ICAM-3) cytokine receptors (e.g., interleukin-4R, interleukin-5R, interleukin-6R, interleukin-9R, interleukin-10R, interleukin-12R, interleukin-13Ralpha1, interleukin-13Ralpha2, interleukin-15R, interleukin-21Ralpha), chemokines, cell death-inducing signal molecules (e.g., B7-H1, DR6 (Death receptor 6), PD-1 (Programmed death-1), TRAIL R1), costimulating molecules (e.g., B7-1, B7-2, B7-H2, ICOS (inducible co-stimulator)), growth factors (e.g., ErbB2, ErbB3, ErbB4, HGFR), differentiation-inducing factors (e.g., B7-H3), activating factors (e.g., NKG2D), signal transfer molecules (e.g., gpl30), BCMA, and TACI.

### Methods of Making Antibodies and Antibody Derivatives

Antibodies and derivatives thereof that are useful in the present methods can be produced by recombinant expression techniques, from hybridomas, from myelomas or from other antibody expressing mammalian cells. Recombinant expression of an antibody or derivative thereof that binds to a target antigen typically involves construction of an expression vector containing a nucleic acid that encodes the antibody or derivative thereof. Once a nucleic acid encoding such a protein has been obtained, the vector for the production of the protein molecule may be produced by recombinant DNA technology using techniques well known in the art. Standard techniques such as those described in Sambrook and Russell, Molecular Cloning: A Laboratory Manual (Cold Spring Harbor Laboratory Press, Cold Spring Harbor, N.Y., 3rd ed., 2001); Sambrook et al., Molecular Cloning: A Laboratory Manual (Cold Spring Harbor Laboratory Press, Cold Spring Harbor, N.Y., 2nd ed., 1989); Ausubel et al., Short Protocols in Molecular Biology (John Wiley & Sons, New York, 4th ed., 1999); and Glick & Pasternak, Molecular Biotechnology: Principles and Applications of Recombinant DNA (ASM Press, Washington, D.C., 2nd ed., 1998) can be used for recombinant nucleic acid methods, nucleic acid synthesis, cell culture, transgene incorporation, and recombinant protein expression.

For example, for recombinant expression of antibody, an expression vector may encode a heavy or light chain thereof, or a heavy or light chain variable domain, operably linked to a promoter. An expression vector may include, e.g., the nucleotide sequence encoding the constant region of the antibody molecule (see, e.g., WO 86/05807; WO 89/01036; and U.S. Pat. No. 5,122,464), and the variable domain of the antibody may be cloned into such a vector for expression of the entire heavy or light chain. The expression vector is transferred to a host cell by techniques known in the art, and the transfected cells are then cultured by techniques known in the art in the presence of a fucosylation inhibitor to produce the antibody. Typically, for the expression of double-chained antibodies, vectors encoding both the heavy and light chains can be co-expressed in the host cell for expression of the entire immunoglobulin molecule.

A variety of mammalian cells and cell lines can be utilized to express an antibody or derivative thereof. For example, mammalian cells such as Chinese hamster ovary cells (CHO) (e.g., DG44, Dxb11, CHO-K, CHO-K1 and CHO-S) can be used. In some embodiments, human cell lines are used. Suitable myeloma cell lines include SP2/0 and IR983F and human myeloma cell lines such as Namalwa. Other suitable cells include human embryonic kidney cells (e.g., HEK293), monkey kidney cells (e.g., COS), human epithelial cells (e.g., HeLa), PERC6, Wil-2, Jurkat, Vero, Molt-4, BHK, and K6H6. Other suitable host cells include YB2/0 cells. In other embodiments, the host cells are not YB2/0 cells.

In some embodiments, the host cells are from a hybridoma. In some embodiments, the host cells are not a hybridoma produced by a fusion generated with NS0 myeloma cells. In other embodiments, the host cells are not from a hybridoma.

In some embodiments, the host cells do not contain a fucose transporter gene knockout. In some embodiments, the host cells do not contain a fucosyltransferase (e.g., FUT8) gene knockout. In some embodiments, the host cells do not contain a knock-in of a GnTIII encoding nucleic acid. In some embodiments, the host cells do not contain a knock-in of a golgi alpha mannosidase II encoding nucleic acid.

A variety of mammalian host-expression vector systems can be utilized to express an antibody or derivative thereof. For example, mammalian cells such as Chinese hamster ovary cells (CHO) (e.g., DG44, Dxb11, CHO-K1 and CHO-S) in conjunction with a vector such as the major intermediate early gene promoter element from human cytomegalovirus or the Chinese hamster ovary EF-1α promoter, is an effective expression system for the production of antibodies and derivatives thereof (see, e.g., Foecking et al., 1986, Gene 45:101; Cockett et al., 1990, Bio/Technology 8:2; Allison, U.S. Pat. No. 5,888,809).

The cell lines are cultured in the appropriate culture medium. Suitable culture media include those containing, for example, salts, carbon source (e.g., sugars), nitrogen source, amino acids, trace elements, antibiotics, selection agents, and the like, as required for growth. For example, commercially available media such as Ham's FlO (Sigma), Minimal Essential Medium (MEM, Sigma), RPMI-1640 (Sigma), Dulbecco's Modified Eagle's Medium ((DMEM, Sigma), PowerCHO™ cell culture media (Lonza Group Ltd.) Hybridoma Serum-Free Medium (HSFM) (GIBCO) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, can be those previously used with the host cell selected for expression, and will be apparent to the ordinarily skilled artisan.

The cells expressing the antibody or antibody derivative can be cultured by growing the host cell in any suitable volume of culture media. The cells may be cultured in any suitable culture system and according to any method known in the art, including T-flasks, spinner and shaker flasks, WaveBag® bags, roller bottles, bioreactors and stirred-tank bioreactors. Anchorage-dependent cells can also be cultivated on microcarrier, e.g., polymeric spheres, that are maintained in suspension in stirred-tank bioreactors. Alternatively, cells can be grown in single-cell suspension. Culture medium may be added in a batch process, e.g., where culture medium is added once to the cells in a single batch, or in a fed batch process in which small batches of culture medium are periodically added. Medium can be harvested at the end of culture or several times during culture. Continuously perfused culture processes are also known in the art, and involve continuous feeding of fresh medium into the culture, while the same volume is continuously withdrawn from the reactor. Perfused cultures generally achieve higher cell densities than batch cultures and can be maintained for weeks or months with repeated harvests.

For cells grown in batch culture, the volume of culture medium is typically at least 750 mL, 1 liter, 2 liters, 3 liters, 4 liters, 5 liters, 10 liters, 15 liters, 20 liters or more. For industrial applications, the volume of the culture medium can be at least 100 liters, at least 200 liters, at least 250 liters, at least 500 liters, at least 750 liters, at least 1000 liters, at least 2000 liters, at least 5000 liters or at least 10,000 liters.

In some embodiments, antibodies or antibody derivatives produced by the instant methods comprise at least 10%, at least 20%, at least 30%, at least 40% or at least 50% non-core fucosylated protein (e.g., lacking core fucosylation). In some embodiments, antibodies or antibody derivatives produced by the instant methods comprise at least 60%, at least 70%, at least 75%, at least 80%, at least 85%, at least 90% or at least 95% non-core fucosylated antibody or antibody derivative. In some embodiments, a composition of antibodies or antibody derivatives produced by the instant methods comprises less than 100% non-core fucosylated antibodies and/or antibody derivatives.

The content (e.g., the ratio) of sugar chains in which fucose is not bound to N-acetylglucosamine in the reducing end of the sugar chain versus sugar chains in which fucose is bound to N-acetylglucosamine in the reducing end of the sugar chain can be determined according to any method known in the art. Such methods include hydrazinolysis or enzyme digestion (see, e.g., Biochemical Experimentation Methods 23: Method for Studying Glycoprotein Sugar Chain (Japan Scientific Societies Press), edited by Reiko Takahashi (1989)), fluorescence labeling or radioisotope labeling of the released sugar chain and then separating the labeled sugar chain by chromatography. Also, the compositions of the released sugar chains can be determined by analyzing the chains by the HPAEC-PAD method (see, e.g., J. Liq Chromatogr. 6:1557 (1983)). (See generally U.S. Patent Application Publication No. 2004-0110282.)

In some embodiments, the antibodies or antibody derivatives produce by the instant methods have higher effector function (e.g., ADCC activity) than the antibodies or antibody derivatives produced in the absence of a fucosylation inhibitor. The effector function activity may be modulated by controlling the level of afucosylation according to any of the methods described herein. ADCC activity may be measured using assays known in the art and in exemplary embodiments increases by at least 10%, 20%, 30%, 40%, 50%, 60%, 70%, 80%, 90%, 2-fold, 3-fold, 4-fold, 5-fold, 6-fold, 7-fold, 8-fold, 9-fold, 10-fold, 15-fold or 20-fold, as compared to the core fucosylated parent antibody. The cytotoxic activity against an antigen-positive cultured cell line can be evaluated by measuring effector function (e.g., as described in Cancer Immunol. Immunother. 36:373 (1993)).

Antibodies and antibody derivative can be purified using, for example, hydroxylapatite chromatography, gel electrophoresis, dialysis, and affinity chromatography, with affinity chromatography being a preferred purification technique. The suitability of protein A as an affinity ligand depends on the species and isotype of any immunoglobulin Fc domain that is present in the antibody or antibody derivative. Protein A can be used to purify antibodies or antibody derivatives that are based on human IgG1, 2, or 4 heavy chains.

Protein G can be used for mouse isotypes and for some human antibodies and antibody derivatives. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody or antibody derivative comprises a C_{H}3 domain, the Bakerbond ABXT™ resin (J. T. Baker, Phillipsburg, N.J.) is useful for purification. Other techniques for protein purification such as fractionation on an ion-exchange column (cationic or anionic exchange), ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody or antibody derivative to be recovered.

Following any purification step(s), the mixture comprising the antibody or antibody derivative of interest and contaminants may be subjected to low pH hydrophobic interaction chromatography (e.g., using an elution buffer at a pH between about 2.5-4.5, preferably performed at low salt concentrations (e. g., from about 0-0.25 M salt)).

### Uses of the Antibodies and Antibody Derivatives

Antibodies and antibody derivatives prepared according to the present methods can be used for a variety of therapeutic and non-therapeutic applications. For example, the antibodies can be used as therapeutic antibodies. Antibody derivatives (e.g., a receptor-Fc fusion) can be used as a therapeutic molecule. In some embodiments, the antibody or antibody derivative is not conjugated to another molecule. In some embodiments, the antibody is conjugated to a suitable drug (e.g., an antibody drug conjugate) or other active agent. The antibodies and antibody derivatives can also be used for non-therapeutic purposes, such as diagnostic assays, prognostic assays, release assays and the like.

### Pharmaceutical Compositions.

Antibodies and antibody derivatives prepared according to the present methods can be formulated for therapeutic and non-therapeutic applications. The antibodies and derivatives can be formulated as pharmaceutical compositions comprising a therapeutically or prophylactically effective amount of the antibody or derivative and one or more pharmaceutically compatible (acceptable) ingredients. For example, a pharmaceutical or non-pharmaceutical composition typically includes one or more carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable excipients include, for example, amino acids, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin. Such compositions will typically contain a therapeutically effective amount of the protein, typically in purified form, together with a suitable amount of carrier so as to provide the form for proper administration to the patient. The formulations correspond to the mode of administration.

Typically, compositions for intravenous administration are solutions in sterile isotonic aqueous buffer. When necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. When the pharmaceutical is to be administered by infusion, it can be dispensed with an infusion bottle containing sterile pharmaceutical grade water or saline. When the pharmaceutical is administered by injection, an ampoule of sterile water for injection or saline can be provided so that the ingredients can be mixed prior to administration.

The invention is further described in the following examples, which are not intended to limit the scope of the invention.

### EXAMPLES

### Example 1: Dependence of mAb % Afucosylation on 2FF Concentration and ICA

Industrial-relevant Chinese hamster ovary (CHO) cell lines were used in this study. The cell lines were derived from a dihydrofolate minus (dhfr-) CHO host (Urlaub G, Chasin L A, Isolation of Chinese hamster cell mutants deficient in dihydrofolate reductase activity. Proc Natl Acad Sci USA 77:4216-4220, 1980). Cells were cultured and maintained in a shake flask using industry-standard proprietary chemically-defined basal medium. The shake flask culture conditions were 37° C., 5% CO₂ through the scale up and end of culturing. Industrial-standard proprietary basal and feed media were used to culture the cells. Variable feed volumes were added to the culture. The glucose concentration was maintained throughout the culture.

2-fluorofucose (2FF) was added in cell culture medium from 10-100 mM stock solutions at the start of the cell culturing process. Multiple concentrations ranging from 0 to 100 μM were tested for multiple cell lines producing different antibody sequences. Daily samples of 1 ml were taken for the entire duration of culture to monitor the cell culture process and to measure viable cell density (VCD) using an automated cell counter. FIG. 1A shows representative growth curves for an exemplary cell line tested. At the end of culturing, cell culture fluid was harvested, centrifuged, and purified using a Protein-A chromatography method. Glycosylation was measured on the protein-A purified samples using a HILIC (Hydrophobic Interaction Chromatography) assay to quantify % afucosylated species in the mAb as a ratio.

### Tuning Using 2FF Concentration and Cell Density

The consumption rate of 2FF (ratio of 2FF concentration over integral cell area (ICA), the area under the viable cell density curve) was estimated with the range of concentrations being tested. % Afucosylation was plotted against the [2FF concentration]/ICA to generate a single saturation curve for afucosylation for multiple cell lines (see FIG. 1B). This curve unifies the prediction of afucosylation across multiple cell lines. This curve was fit to a Michaelis-Menten kinetics equation to determine the constants in the equation.

As shown in FIG. 1B, a saturation limit was observed beyond which additional 2FF would not increase the afucosylation. This empirical model enables the estimation of afucosylation for a particular cell line if its ICA is known and unifies the relationship across multiple CHO cell lines. In addition, the model provides a tool to tune afucosylation to achieve either full or partial saturation.

### Example 2: Dependence of mAb % Afucosylation on Time of Addition of Saturating 2FF and Antibody Titer at Time of Saturating 2FF Addition

Industrial-relevant Chinese hamster ovary (CHO) cell lines were used in this study. The cell lines were derived from a dihydrofolate minus (dhfr-) CHO host (Urlaub G, Chasin L A, Isolation of Chinese hamster cell mutants deficient in dihydrofolate reductase activity. Proc Natl Acad Sci USA 77:4216-4220, 1980). Cells were cultured and maintained in a shake flask using industry-standard proprietary chemically-defined basal medium. The shake flask culture conditions were 37° C., 5% CO₂ through the scale up and end of culturing. Industrial-standard proprietary basal and feed media were used to culture the cells and variable feed volumes were added to the culture. The glucose concentration was maintained throughout the culture.

### Tuning Using Time of 2FF Addition

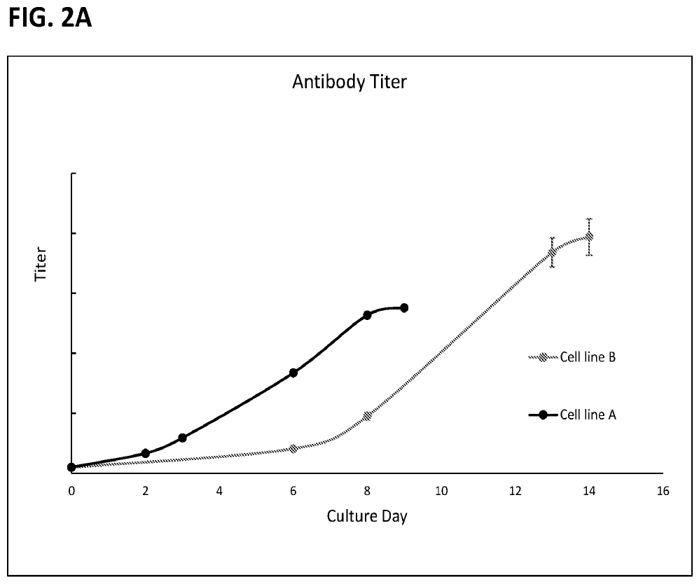

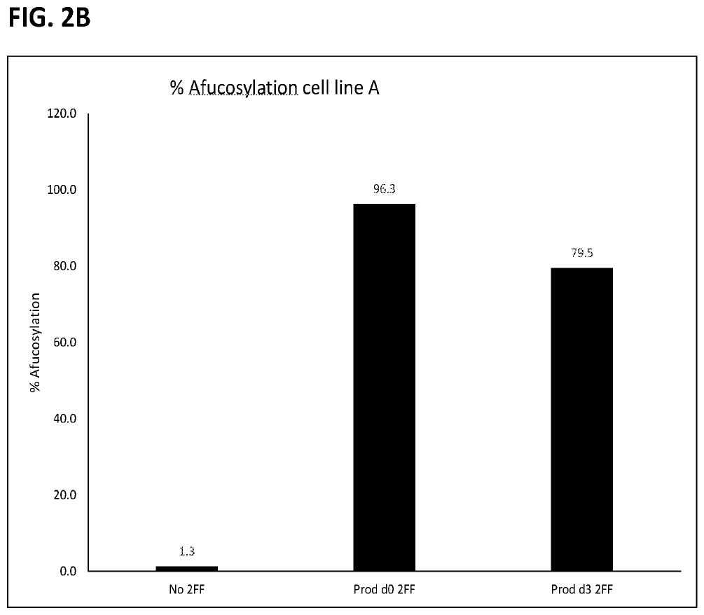

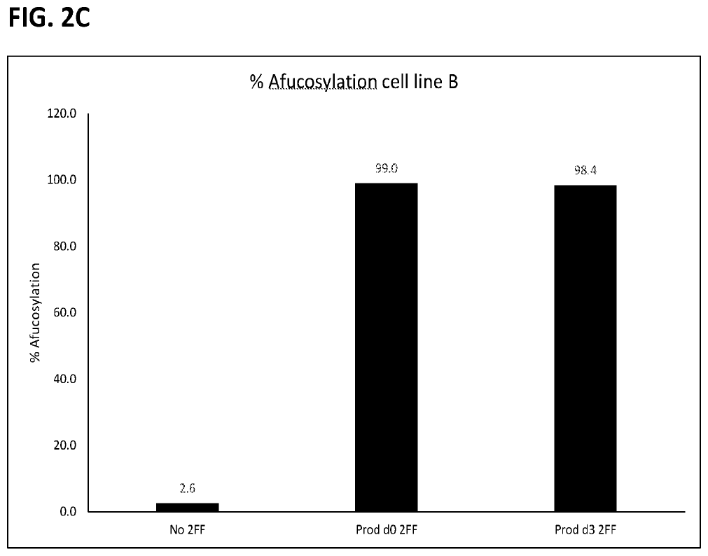

The effect of timing of addition of the fucosylation inhibitor 2FF on afucosylation was tested using two industrially relevant CHO cell lines (cell line A and cell line B) producing different antibodies. As shown in FIG. 2A, the antibody production curves for the 2 cell lines have different kinetics. 2FF was added in the cell culture medium from a 100 mM stock to reach a final concentration of 100 μM. Addition was performed either on day 0 or day 3 of the culturing process. At the end of culturing, cell culture fluid was harvested, centrifuged and purified using a Protein-A chromatography method. Samples were analyzed for afucosylation levels using the HILIC assay as described in Example 1. For cell line A, which produced a significant fraction of antibody by day 3, partial afucosylation was observed (FIG. 2B), unlike for cell line B (FIG. 2C), which did not produce a significant fraction of the final day antibody titer by day 3. This empirical model enables the estimation of afucosylation for a particular cell line if its antibody production curve is known.

The present invention is not limited in scope by the specific embodiments described herein. Various modifications of the invention in addition to those described herein will become apparent to those skilled in the art from the foregoing description and accompanying figures. Such modifications are intended to fall within the scope of the appended claims. Unless otherwise apparent from the context any step, element, embodiment, feature or aspect of the invention can be used in combination with any other. All patent filings, and scientific publications, accession numbers and the like referred to in this application are hereby incorporated by reference in their entirety for all purposes to the same extent as if so individually denoted.

What is claimed is:

1. A method of controlling the level of afucosylation of an antibody or antibody derivative, comprising:
(a) culturing a host cell in a culture medium in the presence of a pre-determined amount of an inhibitor of fucosylation (Aₚ), wherein the host cell expresses an antibody or antibody derivative having an Fc domain having at least one complex N-glycoside-linked sugar chain bound to the Fc domain through an N-acetylglucosamine of the reducing terminal of the sugar chain; and
(b) isolating the antibody or antibody derivative,
wherein Aₚ is pre-determined such that the level of afucosylation of the isolated antibody or antibody derivative of (b) has a level of afucosylation that does not exceed a maximum deviation from a target level of afucosylation.

2. The method of claim 1, wherein the antibody or antibody derivative is isolated upon completion of culturing.

3. The method of claim 1 or 2, further comprising determining Ap.

4. The method of any one of claims 1-3, wherein Ap is determined based on a predictive model generated using a plurality of different fucosylation inhibitor amounts and a cell growth parameter of the host cell in the culture as inputs and the level of afucosylation of the isolated antibody or antibody derivative as the output.

5. The method of claim 4, wherein the predictive model is generated using fucosylation inhibitor amounts normalized to the cell growth parameter as inputs.

6. The method of claim 4 or 5, wherein the cell growth parameter is integral cell area (ICA).

7. The method of any one of claims 4-6, further comprising generating the predictive model.

8. The method of any one of claims 1-7, wherein the fucosylation inhibitor is a fucose analog.

9. The method of claim 8, wherein the fucose analog is 2-fluorofucose (2FF), the compound of formula I, or the compound of formula II.

10. The method of claim 9, wherein the fucose analog is 2FF.

11. The method of any one of claims 1-10, wherein the target level of afucosylation is:
(a) about 100% to about 90%;
(b) about 90% to about 80%;
(c) about 80% to about 70%;
(d) about 70% to about 60%;
(e) about 60% to about 50%;
(f) about 50% to about 40%;
(g) about 40% to about 30%;
(h) about 30% to about 20%;
(i) about 20% to about 10%; or
(j) about 10% to about 0%.

12. The method of any one of claims 1-10, wherein the target level of afucosylation is:
(a) greater than about 80%;
(b) greater than about 60%;
(c) greater than about 40%;
(d) greater than about 20%;
(e) greater than about 10%; or
(f) greater than about 5%.

13. The method of any one of claims 1-12, wherein the maximum deviation from the target level of afucosylation is no more than 10%.

14. The method of claim 13, wherein the maximum deviation from the target level of afucosylation is no more than 5%.

15. A method of controlling the level of afucosylation of an antibody or antibody derivative, comprising:
(a) culturing a host cell in a culture medium, wherein the host cell expresses an antibody or antibody derivative having an Fc domain having at least one complex N-glycoside-linked sugar chain bound to the Fc domain through an N-acetylglucosamine of the reducing terminal of the sugar chain;
(b) adding a saturating amount of an inhibitor of fucosylation to the culture medium at a pre-determined time (Tp) during the culturing, wherein the saturating amount of the fucosylation inhibitor results in at least about 95% afucosylation when added at d0 of the culturing; and
(c) isolating the antibody or antibody derivative,
wherein Tp is pre-determined such that the level of afucosylation of the isolated antibody or antibody derivative of (c) has a level of afucosylation that does not exceed a maximum deviation from a target level of afucosylation.

16. The method of claim 15, wherein the antibody or antibody derivative is isolated upon completion of culturing.

17. The method of claim 15 or 16, further comprising determining Tp.

18. The method of any one of claims 15-17, wherein Tp is determined based on a predictive model generated using titer of the antibody or antibody derivative in the culture at a plurality of different saturating fucosylation inhibitor addition times in the culturing as inputs and the level of afucosylation of the isolated antibody or antibody derivative as the output.

19. The method of claim 18, further comprising generating the predictive model.

20. The method of any one of claims 15-19, wherein the fucosylation inhibitor is a fucose analog.

21. The method of claim 20, wherein the fucose analog is 2FF, the compound of formula I, or the compound of formula II.

22. The method of claim 21, wherein the fucose analog is 2FF.

23. The method of any one of claims 15-22, wherein the target level of afucosylation is:
(a) about 100% to about 90%;
(b) about 90% to about 80%;
(c) about 80% to about 70%;
(d) about 70% to about 60%;
(e) about 60% to about 50%;
(f) about 50% to about 40%;
(g) about 40% to about 30%;
(h) about 30% to about 20%;
(i) about 20% to about 10%; or
(j) about 10% to about 0%.

24. The method of any one of claims 15-22, wherein the target level of afucosylation is:
(a) greater than about 80%;
(b) greater than about 60%;
(c) greater than about 40%;
(d) greater than about 20%;
(e) greater than about 10%; or
(f) greater than about 5%.

25. The method of any one of claims 15-24, wherein the maximum deviation from the target level of afucosylation is no more than 10%.

26. The method of claim 25, wherein the maximum deviation from the target level of afucosylation is no more than 5%.

27. The method of any one of claims 1-26, wherein the host cell is a recombinant host cell.

28. The method of claim 27, wherein the host cell is a Chinese hamster ovary (CHO) cell.

29. The method of any one of claims 1-26, wherein the host cell is a hybridoma.

30. The method of any one of claims 1-29, wherein the host cell is grown in fed batch culture.

31. The method of any one of claims 1-29, wherein the host cell is grown in continuous feed culture.

32. The method of any one of claims 1-31, wherein the culture medium has a volume of at least 100 liters.

33. The method of claim 32, wherein the culture medium has a volume of at least 500 liters.

34. The method of any one of claims 1-33, wherein the culture media is an animal protein free media.

35. The method of any one of claims 1-34, wherein isolating the antibody or antibody derivative comprises isolating the antibody or antibody derivative from the cell and/or the culture medium.

36. The method of claim 35, wherein isolating the antibody or antibody derivative comprises using a protein A column.

37. The method of claim 35, wherein isolating the antibody or antibody derivative comprises using a cation or anion exchange column or a hydrophobic interaction column.

38. The method of any one of claims 1-37, wherein the antibody or antibody derivative is an intact antibody.

39. The method of claim 38, wherein the intact antibody is an IgG1 antibody.

40. The method of any one of claims 1-37, wherein the antibody or antibody derivative is a single chain antibody.

41. The method of any one of claims 1-37, wherein the antibody or antibody derivative comprises a heavy chain variable region, a light chain variable region, and an Fc region.

42. The method of any one of claims 1-37, wherein the antibody or antibody derivative is an antibody derivative comprising an antibody Fc region and a ligand binding domain of a non-immunoglobulin protein.
